SEQUENCE LISTING (5 sequences)

SEQ ID NO: 1 - DNA, 18 bp, Artificial Sequence
  Description of Artificial Sequence Synthetic primer
tgcacgcaccgtgtactc

SEQ ID NO: 2 - DNA, 18 bp, Artificial Sequence
  Description of Artificial Sequence Synthetic primer
agtggacacgttggtcgg

SEQ ID NO: 3 - DNA, 15 bp, Artificial Sequence
  Description of Artificial Sequence Synthetic oligonucleotide
gggattaattggagg

SEQ ID NO: 4 - DNA, 51 bp, Artificial Sequence
  Description of Artificial Sequence Synthetic oligonucleotide
tgcacgcaccgtgtactcgggattaattggaggagtggacacgttggtcgg

SEQ ID NO: 5 - DNA, 18 bp, Artificial Sequence
  Description of Artificial Sequence Synthetic primer
ccgaccaacgtgtccact

(19) United States
(12) Patent Application Publication
(10) Pub. No.: US 2006/0088861 A1
(43) Pub. Date: Apr. 27, 2006

(54) Information nucleic acid-carrying fine particles and production method thereof

(75) Inventors: Tsunehiko Higuchi, Nagoya (JP); Hidetoshi Hayashi, Nagoya (JP); Hiroshi Yokoyama, Kanagawa (JP); Masahiko Yamanaka, Kanagawa (JP); Kentarou Watanabe, Kanagawa (JP)

Correspondence Address: FOLEY AND LARDNER LLP;SUITE 500, 3000 K STREET NW, WASHINGTON, DC, 20007, US

(73) Assignees: NISSAN MOTOR CO., LTD.; Tsunehiko HIGUCHI

(21) Appl. No.: 11/236,843

(22) Filed: Sep. 28, 2005

(30) Foreign Application Priority Data
Sep. 30, 2004 (JP) 2004-286555

(51) Int. Cl. C12Q 1/68; B05D 3/00

(52) U.S. Cl. 435/6; 427/2.11

(57) ABSTRACT

Information nucleic acid-carrying fine particles to be used for individuality authentication for industrial products. The information nucleic acid-carrying fine particles include fine particles on which an information nucleic acid including a base sequence portion having an arbitrary and known base sequence is carried.

### BACKGROUND OF THE INVENTION

This invention relates to improvements in an individuality authentication technique for a variety of articles, and more specifically to an information nucleic acid-carrying fine particles in which an information nucleic acid can be stably and uniformly dispersed when the information nucleic acid used as an origin and history information for an industrial product or the like is added to the product while preventing the information nucleic acid from flowing out from the product for a long period of time, and a method of producing such information nucleic acid-carrying fine particles.

Hitherto, in order to authenticate an individuality of articles, a license plate, a watermarking for paper money, an IC chip, a facial portrait for a credit card and the like have been employed as an individuality authenticating means. However, these individuality authenticating means have encountered such drawbacks as to be removable from a product, for example, by being peeled off, cut out and erased. Accordingly, it has been desired to develop an authentication information which cannot be removed or vanished from the product.

In this regard, DNA inherently contained in every organism is an information biopolymer including all genetic information of the organism. Most DNA correspond to many amino acid sequences of protein. DNA includes compounds such as deoxyadenosine (dA), deoxyguanosine (dG), deoxycytosine (dC) and thymidine (dT) which are bonded in a certain direction through phosphoric ester links. Assuming that the number of bases of DNA is n, 4ⁿ kinds of DNA will exist. Accordingly, the existence of about 4.3 billions kinds of distinguishable DNA is assumed even resulting from only 16 kinds of bases. At the present time, in synthesis of DNA having several tens of base sequences, any DNA having any base can be freely synthesized. In addition, concerning DNA in an amount more than a certain level, its base sequence can be automatically determined by an automatic sequence reader or sequencer.

As the above techniques using DNA, the following proposition has been made as disclosed in Japanese Patent Provisional Publication No. 2004-159502 in which a product is provided with a counterfeit-proof label made of a water-insoluble medium containing DNA. The authenticity of the product can be checked according to the presence or absence of DNA.

### SUMMARY OF THE INVENTION

However, the technique or proposition disclosed in Japanese Patent Provisional Publication No. 2004-159502 basically relates to a method for mixing DNA with the water-insoluble medium. As a method for checking the authenticity of the product as to whether the product is true or false, the publication only discloses that the target product containing ribonucleic acid is identified by detecting whether ribonucleic acid is amplified or not by using a PCR method. Additionally, the publication never discloses individuality authentication data using presence or absence of DNA as an examination index as well as data which relate to individuality authentication and makes possible the individuality authentication of each product even in the same kind of products.

By the way, it has been required to develop such a technique that information relating to the origin and history of an industrial product is supplied as much as possible to the industrial product, and it can be authenticated as occasion demands. An example of the above technique is to establish the following individuality authentication manner: In an automotive accident where an assaulter has run away, the origin and history of an accidental vehicle is individually authenticated according to paint pieces or plastic pieces left on the accidental scene thereby specifying an objective vehicle.

However, in case that nucleic acid represented by DNA having a known base sequence is used for individuality authentication for the above products, the nucleic acid is water-soluble, and therefore there is the possibility that the nucleic acid flows out from the product if it is combined in the product so that the nucleic acid is difficult to be maintained in the product throughout a long period of time.

In view of the above, it is an object of the present invention to provide improved information nucleic acid-carrying fine particles and production method of the fine particles, by which drawbacks encountered in conventional authentication techniques using DNA can be overcome.

Another object of the present invention is to provide improved information nucleic acid-carrying fine particles and production method of the fine particles, by which the information nucleic acid which has a known base sequence to be used for authentication of the origin and history of a product can be stably left or maintained in the product for a long period of time thereby making it possible to authenticate the origin and history of the product for a long period of time.

An aspect of the present invention resides in information nucleic acid-carrying fine particles comprising fine particles on which an information nucleic acid including a base sequence portion or site having an arbitrary and known base sequence is carried.

Another aspect of the present invention resides in a method of producing information nucleic acid-carrying fine particles. The method comprises (a) adding sterilized distilled water to the fine particles to prepare a suspension; (b) adding to the suspension at least one of the information nucleic acid and a solution prepared by dissolving the information nucleic acid to sterilized distilled water; and (c) drying the suspension to which at least one of the information nucleic acid and the solution.

### BRIEF DESCRIPTION OF THE DRAWING

FIG. 1A is a structural formula of a natural type DNA;

FIG. 1B is a structural formula in which a hydroxyl group at position 5′ of DNA of FIG. 1A is derivatized;

FIG. 2 is a schematic view of a single-stranded DNA whose identification information site is provided at its both ends with primer binding sites, as an example of an information nucleic acid to be used in the present invention; and

FIG. 3 is a fragmentary schematic sectional view of a specimen to be tested which specimen contains information nucleic acid-carrying fine particles according to the present invention.

### DETAILED DESCRIPTION OF THE INVENTION

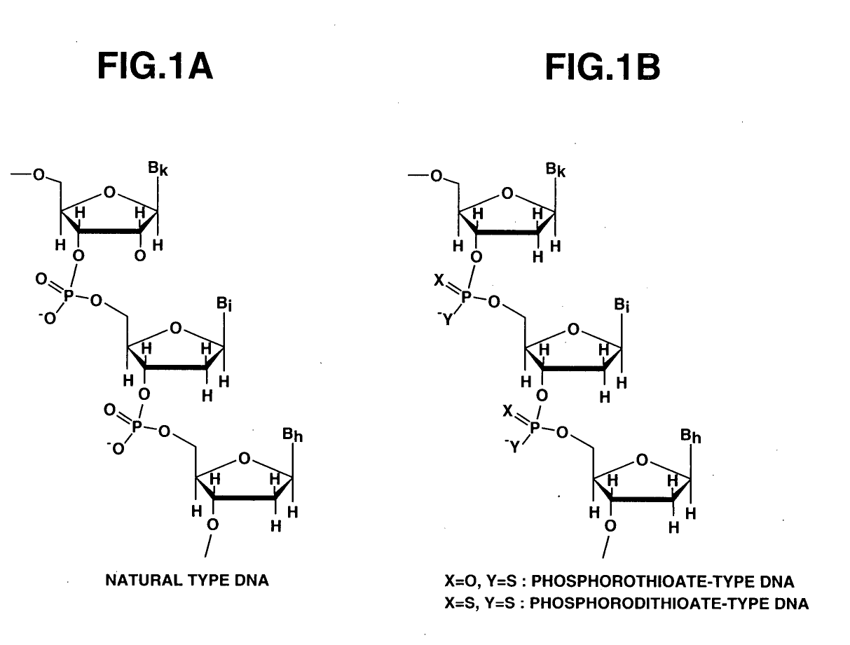

Referring now to FIGS. 1A, 1B, 2 and 3 of the drawings, information nucleic acid-carrying fine particles according to the present invention will be discussed. The information nucleic acid-carrying fine particles comprise fine particles on which an information nucleic acid including a base sequence portion or site having an arbitrary and known base sequence is carried. In other words, in the information nucleic acid-carrying fine particles, the information nucleic acid is carried on the fine particles such as silica fine particles or zinc oxide (zinc while) fine particle. By using the information nucleic acid-carrying fine particles, it is made possible to stably add the information nucleic acid to a product while preventing the information nucleic acid from flowing out under the influence of a water content.

The state of the information nucleic acid being carried on the fine particles means a state in which the information nucleic acid is in tight contact with the fine particles in such a degree that the information nucleic acid can move together with the fine particles in a state to be put at the surface of the fine particles. Accordingly, the information nucleic acid may be firmly adhered to the surface of the fine particles, or may be attached to the surface of the fine particles or to the inner surface of depression formed in the fine particles with such a strength as to be durable in use.

The information nucleic acid in the present invention includes the portion or site having the arbitrary and known base sequence which portion hereinafter referred to as a base sequence portion or site. The information nucleic acid can be easily contained in a product and the material of the product while being usable as an individuality authenticating means which is difficult to be removed from the product.

The information nucleic acid includes DNA (deoxyribonucleic acid), RNA (ribonucleic acid) and derivatives of DNA and RNA. Although either a natural type nucleic acid or an artificial type nucleic acid can be used, it is preferable to use the artificial type one which is structurally stable taking account of causing it to be contained in the product used under a severe condition. In the artificial type nucleic acid, a base sequence which does not exist in the natural type nucleic acid can be formed.

In the information nucleic acid, the arbitrary base sequence of the information nucleic acid means that the base sequence can be freely selected as far as it is detectable or readable. The known base sequence means that the base sequence used for individuality authentication has been previously grasped or ascertained.

Regarding the size of the information nucleic acid, it is preferable that the number of bases in the whole of the information nucleic acid is not larger than 200. In case where the number of the bases is larger than 200, unreacted portions or sites are made bit by bit at the stage of synthesis so that a content of nucleic acids whose bases are missed is liable to increase. It is more preferable that the number of the bases is about 100.

Further, it is preferable that thymine and thymine are not adjacent to each other in the above base sequence from the view point of suppressing dimerization of thymines. Further, it is preferable that the information nucleic acid is derivatized with a protective group from the view points of improving stability in cases where it is used together with a compound which can react with a hydroxyl group and used under the severe condition. Specifically, at least one of hydroxyl groups at positions 3′ and 5′ can be derivatized with a phosphoric ester group, an acyl group, an alkoxycarbonyl group, a benzyl group, a substituted benzyl group, an allyl group and the like. FIG. 1A shows a structural formula of a natural type DNA, and FIG. 1B shows a structural formula in which hydroxyl group at position 5′ of DNA illustrated in FIG. 1A is derivatized. In FIG. 1B, the illustrated DNA is of a phosphorothioate type in case that X is an oxygen atom and Y is a sulfur atom, and the illustrated DNA is of a phosphorodithioate type in case that both X and Y are sulfur atoms, respectively.

It is further preferable that the hydroxyl group at position 5′ is derivatized with biotin or fluorescent molecules from the viewpoint of improving the convenience in isolation and refinement of the information nucleic acid. Concretely, using biotin to derivatize a part of the information nucleic acid facilitates a selective adsorption of the information nucleic acid to a column to which avidin (a kind of protein) is bonded. On the other hand, using fluorescent molecules such as fluorescein facilitates refinement and the like of the information nucleic acid since nucleic acid itself becomes fluorescent so as to be sensitively detectable. Thus, the improved convenience in isolation and refinement of the information nucleic acid largely facilitates the individuality authentication.

It will be understood that a hydroxyl group at position 2′ may be derivatized with the above-mentioned protective group from the viewpoint of improving stability, when RNA is used as the information nucleic acid.

Furthermore, when the individuality authentication is conducted on the information nucleic acid in a state of being contained in the product, it is preferable that a portion or site used for amplification of the information nucleic acid is the above-mentioned base sequence portion from the viewpoint of achieving an effective detection even in a low content of the information nucleic acid. As a method of the amplification of the information nucleic acid low in content, a polymerase chain reaction (PCR) by which DNA is synergistically amplified can be suitably employed.

In a PCR method using the above PCR, the information nucleic acid even in a very small quantity can be highly amplified. In this PCR method, for example, by acting heat-resistance DNA polymerase on original DNA in the presence of bases or primers complementary to several tens of bases of the original DNA under a temperature control, the original DNA can be amplified. When this operation of amplification is repeated 30 times, the original DNA can be amplified several hundreds of millions times. Such amplification can provide a sufficient amount of DNA to determine the base sequence. As a result, the identity of the product containing the original DNA can be authenticated from the information corresponding the base sequence.

Additionally, in connection with the above, it is preferable that the original DNA has portions (primer binding sites) corresponding to primers at its both ends, as the above-mentioned portion used for the above amplification. The information nucleic acid which does not have the primer may be used; however, provision of the primer can make possible authentication of the original DNA within a short time.

Regarding the primer binding site, it is preferable that the number of the bases is not less than 5, and it is more preferable that the number of the bases is not less than 10. If the number of the bases is less than 5, the number of nucleic acids which are distinguishable is reduced, and therefore much time is necessary to distinguish a lot of target products individually. It is further preferable that the number of the bases is not larger than 100. If the number of the bases is larger than 100, the ratio of by-product missing a base at any position is unavoidably increased. Accordingly, it will take much time and effort to refine, and refinement will become difficult to be done in certain circumstances.

When RNA is used as the information nucleic acid, first DNA complementary in base sequence to the RNA is obtained by using reverse transcriptase, and thereafter the PCR method is carried out to accomplish amplification of the information nucleic acid.

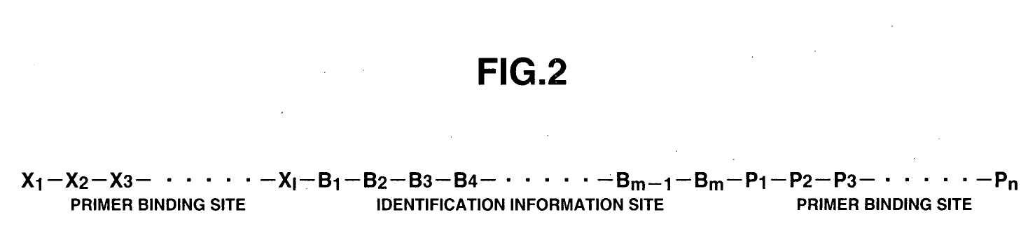

Moreover, it is preferable that the information nucleic acid has an identification information site in addition to the above-mentioned base sequence portion. With this, more detailed information can be set, thereby accomplishing more advanced individual authentication. This is, for example, explained with reference to FIG. 2. As shown in FIG. 2, in case of an information DNA which has the primer binding sites at its both ends, m (number) base sequences (B₁ to Bₘ) are located at the middle, in which the sequence information of this m base sequences correspond to the identification information site. The primer binding sites having 1 (number) and n (number) base sequences (X₁ to X₁) and (P₁ to Pₙ) complementary respectively to 1 and n primers are connected to the both ends. Upon existence of this complementary base sequences, using the PCR method becomes possible for the first time. Either a single-stranded or a double-stranded information DNA can be used as an information element. The double-stranded information DNA is a complex of the single-stranded information DNA and the complementary DNA. The base sequence of the primer binding site can be arranged such that bonds to the complementary base sequence can be stable as much as possible and that the amplification by the PCR method can progress smoothly.

In FIG. 2, each base of X₁ to X₁, B₁ to Bₘ, P₁ to Pₙ is any of deoxyadenosine (dA), deoxyguanosine (dG), deoxycytosine (dC) and thymidine (dT).

In the information nucleic acid-carrying fine particles, the information nucleic acid is carried on the fine particles. Suitable examples of the fine particles are silica, zinc oxide, titanium oxide, molybdenum oxide, tungsten oxide, barium titanate, and the like. The fine particles have an average fine particle diameter (size) preferably within a range of from 0.01 to 60 μm, more preferably within a range of from 0.02 to 5 μm. If the average diameter of the fine particles is less than 0.01 μm, the detection accuracy of the information nucleic acid is lowered. If the average diameter of the fine particles exceeds 60 μm, there arises a tendency to lower the dispersibility of the information nucleic acid-carrying fine particles in a solvent such as sterilized distilled water or alcohol.

In the information nucleic acid-carrying fine particles, the amount of the information nucleic acid carried on the fine particles is preferably within a range of from 0.5 to 10,000 μg, more preferably within a range of from 1.0 to 1000 μg, relative to 100 g of the fine particles. If the amount is less than 0.5 μg relative to 100 g of the fine particles, the information nucleic acid cannot be stably added in the product thereby lowering a detection precision of the information nucleic acid. If the amount exceeds 2000 μg, the information nucleic acid cannot be stably carried on the fine particles, so that free information nucleic acids are unavoidably formed thereby establishing a tendency to make it difficult to effectively disperse the information nucleic acid-carrying fine particles.

The information nucleic acid-carrying fine particles according to the present invention are produced as follows: The above fine particles are dispersed in sterilized distilled water to form a suspension. Then, the above information nucleic acid is added as it is or an information nucleic acid aqueous solution is added to the dispersion, followed by drying. The information nucleic acid aqueous solution has been prepared by dissolving the information nucleic acid in sterilized distilled water. In connection with the above production, it may be allowed that a part of the information nucleic acid is added to the dispersion without becoming in the state of the aqueous solution and the remainder is added to the dispersion upon becoming in the state of the aqueous solution.

In connection with the above production, it is preferable that a solvent(s) is further added to the above suspension. Examples of the solvent are alcohol (for example, methanol, ethanol, propanol, butanol, pentanol, hexanol, heptanol, octanol, nonanol or the like), ester (for example, ethyl acetate, butyl acetate, propyl acetate or the like), ketone (for example, acetone, dimethyl ketone, methyl ethyl ketone, diethyl ketone or the like), and/or aromatic solvent (for example, toluene, hexane, cyclohexane, xylene or the like). This improves the dispersibility of the fine particles in the suspension while promoting the volatilization of water content and solvent content after the information nucleic acid is added to the suspension. It will be understood that the solvent is not limited to one of the above-listed solvents so that two or more of the above-listed solvents are used in combination. Additionally, these solvents may be allowed to be added together with the information nucleic acid, or added after the information nucleic acid is added.

In case that the solvent is alcohol, it is preferable that the amount of the solvent to be added is within a range of from 1 to 99 in volume ratio of sterilized distilled water/the alcohol. In case that the solvent is other than alcohol, i.e., ester, ketone and/or aromatic solvent, it is preferable that the amount of the solvent to be added is within a range of from 1 to 75 in volume ratio of sterilized distilled water/the solvent. In this regard, if the added amount of the solvent is too little, the above-mentioned effects due to addition of the solvent cannot be sufficiently obtained. Conversely, if the added amount is too much, the compatibility of the solvent with water is lowered so that water tends to remain without being volatilization, thereby providing a tendency in which the above-mentioned effects due to the solvent cannot be sufficiently obtained.

### EXAMPLES

The present invention will be more readily understood with reference to the following Example in comparison with Comparative Example; however, these Examples are intended to illustrate the invention and are not to be construed to limit the scope of the invention.

[I] Production of Information Nucleic Acid-Carrying Fine Particles

(1) Synthesis of Information DNA

Nucleosides were sequentially bonded to each other under a phosphoramidite method so that the primer binding sites having the sequences of TGCACGCACCGTGTACTC and AGTGGACACGTTGGTCGG were bonded respectively to the both or opposite sides of the identification information site having the sequence of GGGATTAATTGGAGG thereby synthesizing an information DNA (5′-TGCACGCACCGTGTACTC-GGGATTAATTGGAGG-AGTGGACACGTTGGTCGG-3′) as an information nucleic acid.

(2) Preparation of Suspension

Zinc oxide fine particles provided with silica coating and having an average diameter of 0.02 μm were used as carrier fine particles for carrying the information nucleic acid. The zinc oxide particles were available from Showa Denko K.K. under the trade name of ZS-032. 15 ml of sterilized distilled water containing 30 vol. % of ethanol was added to 4 g of the zinc oxide particles to form a suspension. It will be understood that the silica coating may not be provided to the zinc oxide particles; however, this silica coating will improve the dispersibility of the silica particles in case that the silica particles are mixed with a resin material (plastic) of the like.

(3) Addition of Information DNA

b 10 nmol (164 μg) of the information DNA obtained in the above-mentioned (1) was dissolved in 15 mL of sterilized distilled water to form an information DNA aqueous solution. The information DNA aqueous solution was added to the above suspension obtained in the above-mentioned (2) and sufficiently stirred. Then, 4 mL of ethanol was added to the suspension containing the information DNA aqueous solution, followed by stirring.

(4) Natural Drying

The above suspension (obtained in the above-mentioned (3)) containing the information DNA was poured onto a plastic disc-shaped dish. Then, the dish was covered with air-permeable paper or cloth, and was allowed to stand for 2 days in a state of being covered with the air-permeable paper or cloth so as to be naturally dried.

(5) Vacuum Drying

A nearly dried cake-like material obtained upon natural drying in the above-mentioned (4) was put in a desiccator and vacuum-dried under heating at 40° C. so as to be sufficiently dried.

(6) Pulverization

The sufficiently dried cake-like material was put into a mortar and pulverized to obtain the information nucleic acid-carrying fine particles.

[II] Preparation of Clear Paint

(1) Clear Paint 1 (Example According to the Present Invention)

One gram of information nucleic acid-carrying fine particles obtained by the above production process [1] was added to 100 g of a paint available from Nippon Paint Co., Ltd. under the trade name of Superlac O-130GN3, under stirring. Then, further stirring was made for 1 hour thereby obtaining a clear paint 1 containing the above information DNA together with the carrier fine particles.

(2) Clear Paint 2 (Comparative Example)

Similarly to the preparation of the clear paint 1, 5 μg of the information DNA obtained in the above-mentioned [I]-(1) was added as it was (without being carried on fine particles) to 100 g of the paint available from Nippon Paint Co., Ltd. under the trade name of Superlac O-130GN3, under stirring. Then, further stirring was made for 1 hour thereby obtaining a clear paint 2 containing the information DNA as a single material.

[III] Production of Specimen Provided with Laminated Coating Films

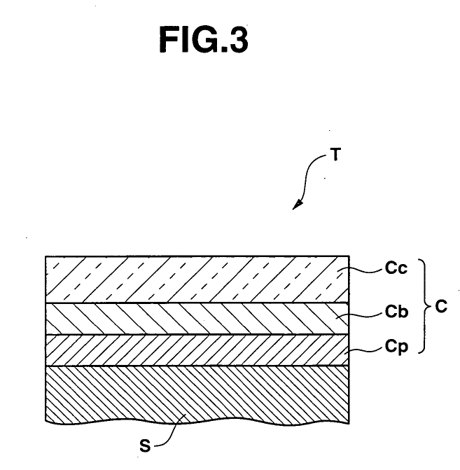

A phosphate-treated dull steel plate S (shown in FIG. 3) having a dimension of 70 mm width, 150 mm length and 8 mm thickness was subjected to an electrodeposition coating in such a manner that a cationic electrodeposition paint (available from Nippon Paint Co., Ltd. under the trade name of Powertop U600M) was coated to form a coating film having a thickness of 20 μm in its dried state. Thereafter, the coated steel plate underwent baking at 160° C. for 30 minutes thereby forming a undercoat layer Cp as a lower-most layer, as shown in FIG. 3. Subsequently, a gray paint available from NOF Corporation under the trade name of Hi-epico No. 500 was coated having a thickness of 30 μm on the coated steel plate, followed by baking at 140° C. for 30 minutes thereby obtained a base coat layer Cb as a middle layer.

Further, the above clear paint 1 was coated on the above base coat layer Cb to have a thickness of 30 μm, followed by baking at 140° C. for 30 minutes thereby forming a clear layer Cc containing the information DNA, as an upper-most layer. Thus, a specimen T was produced having a three-layered laminated coating film C including the layers Cp, Cb and Cc formed on the steel plate S.

Additionally, similarly to the specimen 1, the above clear paint 2 was coated on the above base coat layer Cb to have a thickness of 30 μm, followed by baking at 140° C. for 30 minutes thereby forming a clear layer Cc containing the information DNA, as an upper-most layer. Thus, a specimen T was produced having a three-layered laminated coating film C including the layers Cp, Cb and Cc formed on the steel plate S.

[IV] Detection of DNA Carried Out by Process Including the Following Steps:

(a) Two kinds of the specimens T provided respectively with the clear layers of the clear paint 1 and the clear paint 2 were dipped for 1000 hours in warm water maintained at 50° C. Thereafter, the coating film C of each specimen T was finely fragmentized by using a cutter.

(b) 5 mL of sterilized distilled water was added to the fragmentized coating film and then stirred by using a magnetic stirrer, thereby extracting DNA in a layer of water.

(c) The layer of water was separated from the fragmentized coating film by using a centrifugal separator and then concentrated by a centrifugal evaporator to obtain a concentrated DNA solution.

(d) PCR buffer (5 μL), Taq polymerase (0.25 μL), sterilized distilled water (24.75 μL), first primer (5′-TGCACGCACCGTGTACTC-3′: 5 μL), second primer (5′-CCGACCAACGTGTCCACT-3′: 5 μL) and 2 mM of 2, 3-dideoxynucleosidetriphosphate (5 μL) were mixed to the concentrated DNA solution (5 μL) to obtain a mixed solution.

(e) The mixed solution was heated at 94° C. for 5 minutes and then subjected to repetition of 30 cycles of a temperature control which includes heating at 94° C. for 30 seconds, heating at 40° C. for 30 seconds and heating at 72° C. for 30 seconds in the order mentioned.

(f) The mixed solution was treated at 72° C. for 7 minutes and then preserved at 4° C.

(g) By using a single-stranded DNA splitting enzyme (S1 nuclease which singularly reacted with the single-stranded DNA), excessive primers were split or decomposed. Thereafter, gel filtration was carried out to remove the split primers so as to refine the target double-stranded information DNA.

(h) 2,3-dideoxynucleosidetriphosphate (dNTP) provided with fluorescence and one kind primer (5′-TGCACGCACCGTGTACTC-3′) were mixed to the refined information DNA to obtain a mixture information DNA.

(i) The mixture information DNA was heated at 94° C. for 5 minutes and then subjected to the repetition of 30 cycles of the temperature control as same as in the step (e).

(j) The mixture information DNA was refined by gel filtration and then supplied to an automatic sequencer, so that the base sequence of the information DNA was determined.

(V) Detection Results

As a result of the above, regarding the specimen T provided with the coating film formed using the clear paint 1 containing the information nucleic acid-carrying fine particles according to the present invention, i.e., the coating film including the clear layer Cc containing the information DNA in the state of being carried on zinc oxide fine particles, the sequence of the information DNA contained in the coating film C could be specified. However, regarding the specimen T provided with the coating film formed using the clear paint 2, i.e., the coating film including the clear layer Cc containing the information DNA in a single state, the base sequence of the information DNA could not be specified.

Additionally, it has been confirmed that, in case that the fragmentized coating film is sampled without a long time dipping of the specimen in warm water, it is possible to determine the base sequence of the information DNA contained in the coating film even in the specimen provided with the coating film using the clear paint 2.

As appreciated from the above, according to the present invention, the information nucleic acid including the base sequence portion having the arbitrary and known base sequence is added directly or in the state of a solution prepared by dissolving a part or whole of the information nucleic acid in sterilized distilled water, to a suspension containing fine particles such as silica fine particles or zinc while fine particles, followed by drying. Accordingly, the information nucleic acid can be securely carried at the surface of the above fine particles, so that the information nucleic acid is introduced in a state of being carried on the fine particles, to the product. As a result, the information nucleic acid can be securely added into the product and stably dispersed in the product as compared with a case where the information nucleic acid is introduced in its single state to the product. Additionally, the information nucleic acid can be prevented from flowing out under the influence of water content. Thus, the present invention can provide such extremely excellent effects as to make it possible to achieve the individuality authentication of the origin and history of the product by using the base sequence of the information nucleic acid as a detection index.

The entire contents of Japanese Patent Applications P2004-286555 (filed Sep. 30, 2004) are incorporated herein by reference.

Although the invention has been described above by reference to certain embodiments and examples of the invention, the invention is not limited to the embodiments and examples described above. Modifications and variations of the embodiments and examples described above will occur to those skilled in the art, in light of the above teachings. The scope of the invention is defined with reference to the following claims.

What is claimed is:

1. Information nucleic acid-carrying fine particles comprising:
fine particles; and
an information nucleic acid including a base sequence portion having an arbitrary and known base sequence, the information nucleic acid being carried on fine particles.

2. Information nucleic acid-carrying fine particles as claimed in claim 1, wherein the fine particles have an average particle diameter ranging from 0.01 to 60 μm.

3. Information nucleic acid-carrying fine particles as claimed in claim 1, wherein the information nucleic acid is carried in an amount of 0.5 to 2000 μg relative to 100 of the fine particles.

4. A method of producing information nucleic acid-carrying fine particles, comprising:
adding sterilized distilled water to the fine particles to prepare a suspension;
adding to the suspension at least one of the information nucleic acid and a solution prepared by dissolving the information nucleic acid to sterilized distilled water; and
drying the suspension to which at least one of the information nucleic acid and the solution.

5. A method as claimed in claim 4, further comprising adding at least one selected from the group consisting of alcohol, ester, ketone and aromatic solvent to the suspension.

6. A method as claimed in claim 5, wherein alcohol is added to the suspension in an amount that a volume ratio of sterilized distilled water/alcohol is within a range of from 1 to 99.

7. A method as claimed in claim 5, wherein at least one solvent selected from the group consisting of ester, ketone and aromatic solvent is added to the suspension, in an amount that a volume ratio of sterilized distilled water/the at least one solvent is within a range of from 2 to 99.
